(19) United States
(12) Patent Application Publication
(10) Pub. No.: US 2025/0161398 A1
(43) Pub. Date: May 22, 2025

(54) DESIGN AND DEVELOPMENT OF A NOVEL MESSENGER RNA THERAPEUTIC TO TREAT ATHEROSCLEROSIS

(71) Applicant: UNIVERSITY OF SOUTH FLORIDA, Tampa, FL (US)

(72) Inventor: Hana TOTARY-JAIN, Tampa, FL (US)

(21) Appl. No.: 18/841,332

(22) PCT Filed: Feb. 27, 2023
(86) PCT No.: PCT/US2023/063374

§ 371 Date: Aug. 23, 2024

Related U.S. Application Data
(60) Provisional application No. 63/314,292, filed on Feb. 25, 2022.

(51) Int. Cl. A61K 38/00; A61P 9/10; C12N 15/11; C12N 15/113

(52) U.S. Cl. CPC A61K 38/005; A61P 9/10; C12N 15/11; C12N 15/113; C12N 15/1136; C12N 2310/14; C12N 2320/31

(57) ABSTRACT

Provided are compositions, formulations, and methods for treating vessel stenosis using a polynucleotide.

### CROSS-REFERENCE TO RELATED APPLICATIONS

This application claims priority to U.S. Provisional Application No. 63/314,292 filed on Feb. 25, 2022, the contents of which are incorporated by reference in their entireties.

### STATEMENT REGARDING FEDERALLY SPONSORED RESEARCH

This invention was made with government support under grant number R01 HL 128411 awarded by the National Institutes of Health. The government has certain rights in this invention.

### SEQUENCE LISTING

A Sequence Listing accompanies this application and is submitted as an ASCII text file of the sequence listing named “173738.02462_ST26.xml” which is 1,827 bytes in size and was created on Feb. 27, 2023. The sequence listing is electronically submitted via EFS-Web with the application and is incorporated herein by reference in its entirety.

### BACKGROUND

Atherosclerotic cardiovascular disease (ASCVD) is the leading cause of death in the United States. Intravascular procedures to alleviate vessel stenosis such as percutaneous coronary intervention (PCI) and drug eluting stents (DESs) have greatly improved patient outcomes, but these measures delay endothelial healing and increase the incidence of in stent thrombosis. Although atherosclerosis of the arteries is widely known as a chronic inflammatory condition, targeting this inflammation component with low systemic toxicity remains a clinical struggle. Clinical trials such as CANTOS and RESCUE have transformed the field of CVD research by targeting inflammation to decrease adverse cardiac events, yet the treatment used also caused systemic immune suppression. Despite this progress, site- and cell-selective therapies that specifically target atherosclerotic plaques to inhibit inflammation and proliferation associated with disease causing cells, while protecting the endothelium, are not available.

Therefore, there is a need for compositions and methods for site- and cell-selective therapy that targets both cell proliferation and inflammation that contribute to plaque formation, while preserving the vasculo-protective endothelium.

### SUMMARY

A first aspect of the disclosure is a polynucleotide comprising an RNA molecule which comprises a coding sequence (CDS), a 3′ untranslated region (UTR) and/or a poly(A) tail, a microRNA (miRNA) target sequence, a first arm sequence of a small interfering RNA (siRNA) sequence linked to the 3′ (UTR) or the poly(A) tail by a cleavable linker, and a second arm of the siRNA sequence. In embodiments, the coding sequence CDS encodes a protein. In embodiments, the miRNA is operatively linked to the coding sequence.

Another aspect is a formulation comprising the polynucleotide disclosed above and a carrier operatively configured to deliver the polynucleotide into a cell.

A further aspect is methods of using the disclosed polynucleotide. An aspect of the methods is a method of obtaining a formulation, comprising the disclosed polynucleotide, and administering the formulation to a subject in need thereof. Another aspect is a method comprising obtaining the polynucleotide and incorporating the polynucleotide into a carrier. In embodiments, the polynucleotide incorporated in a carrier is administered to a subject in need thereof, for example as a formulation administered to a subject. One advantage of the claimed methods is that they may be used to prevent, inhibit, or alleviate vessel stenosis.

### BRIEF DESCRIPTION OF THE DRAWINGS

The detailed description includes reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 illustrates microRNA-switches for therapeutic use. Different cell types endogenously produce a unique set of miRNAs that play roles in regulation of gene expression. These naturally occurring miRNAs can be leveraged in mRNA therapeutic design to allow for cell-selective expression of therapeutics.

FIG. 2 illustrates small interfering RNA (siRNA) for therapeutic use. siRNA can be used therapeutically to inhibit expression of disease contributing genes.

FIG. 3A is a drawing of an mi-siRNA that consists of a mRNA polynucleotide that includes a 5′ untranslated region (5′ UTR), a miRNA target region adjacent to or within the 5′ UTR, a coding sequence (CDS), a 3′ untranslated region (3′ UTR), a polyadenylate tail region (poly(A), and a small interfering RNA region (siRNA) containing a first arm and a second complementary DNA-RNA hybrid arm). Asterisks represent dNTPs located at or near the 3′ end of the second arm where RNaseH cleaves and releases the siRNA duplex.

FIG. 3B is a drawing of a mRNA polynucleotide that includes a 5′ untranslated region (5′ UTR), a coding sequence (CDS), a 3′ untranslated region (3′ UTR), a polyadenylate tail region (poly(A), and a small interfering RNA region (siRNA) containing a first arm and a second complementary DNA-RNA hybrid arm). Asterisks represent dNTPs located at or near the 3′ end of the second arm where RNaseH cleaves and releases the siRNA duplex.

FIG. 3C is a drawing of the microRNA switch, consists of a mRNA polynucleotide that includes a 5′ untranslated region (5′ UTR), a miRNA target site upstream of the coding sequence (CDS), a 3′ untranslated region (3′ UTR), and a polyadenylate tail region (poly(A).

FIG. 3D is a drawing of the mi-siRNA, that consists of a mRNA polynucleotide that includes a 5′ untranslated region (5′ UTR), a coding sequence (CDS), a 3′ untranslated region (3′ UTR), a miRNA target region adjacent to or within the 3′ UTR, a polyadenylate tail region (poly(A), and a small interfering RNA region (siRNA) containing a first arm and a second complementary DNA-RNA hybrid arm). Asterisks represent dNTPs located at or near the 3′ end of the second arm where RNaseH cleaves and releases the siRNA duplex.

FIGS. 4A-4C. Schematic mi-siRNA therapeutics to be combined. FIG. 4A is a p27 encoding mRNA that contains a miR-126 switch. FIG. 4B is a p27 encoding mRNA that contains siRNA against IL-1β. FIG. 4C is the mi-siRNA that combines of FIG. 4A and FIG. 4B into a single mRNA-based therapeutic, 126TS-p27-siIL-1β. Red=DNA, Green=RNA. CDS=coding sequence.

FIG. 5A is a microphotograph representing scanning electron microscopy images of p5RHH nanoparticles loaded with near infrared fluorescent protein (niRFP) mRNA on a polycarbonate membrane with 200 nm pores (dark circles).

FIG. 5B are representative immunofluorescent confocal images taken from whole mounts of wire-injured and contralateral control mouse femoral arteries collected 48 h after a single injection of niRFP mRNA-p5RHH nanoparticles. Adapted from Lockhart et al, 2021(1).

FIG. 6A is a diagram illustrating a schematic of RNA molecule RFP-siGFP, with a graph below illustrating RFP signal intensity. The RFP signal intensity is measured via flow cytometry. 293T cells were transfected with miR-126 or miR-145 mimics 24 h before transfection with GFP and RFP-siGFP and cells were collected after 48 h.

FIG. 6B is a diagram illustrating a schematic of mi-siRNA molecule 126TS-RFP-siGFP that includes a combination of miRNA switch and siRNA in one single RNA molecule. A graph illustrating RFP signal intensity is shown below. The RFP signal intensity is measured via flow cytometry. 293T cells were transfected with miR-126 or miR-145 mimics 24 h before transfection with GFP and 126TS-RFP-siGFP and cells were collected after 48 h. *p<0.05 versus miR-145 transfected control.

FIG. 6C is a graph illustrating quantification of GFP signal intensity showing that cells transfected with RFP-mi-siRNA also had decreased GFP expression, comparable to 125 nM of standard siGFP treatment, with a representative immunoblot below.

FIG. 6D is a diagram illustrating a schematic of mi-siRNA molecule 126TS-RFP-siGFP that includes a combination of miRNA switch and siRNA in one single RNA molecule. A representative immunoblot and graph illustrating quantification of GFP normalized to GAPDH is shown below. 293T cells were transfected with miR-126 or miR-145 mimics 24 h before transfection with GFP and 126TS-RFP-siGFP and cells were collected after 48 h.

FIG. 7A are immunofluorescent confocal images illustrating p5RHH-niRFP nanoparticles selectively targeting atherosclerotic plaque regions. Representative confocal en-face images of aorta from ApoE-/- were taken 48 hours after treatment with niRFP mRNA nanoparticles. Images were captured at 60× magnification. Scale bars represents 20 μm. VE-Cadherin to stain ECs, DAPI to stain nuclei. Arrows indicate RFP positive cells at regions of disrupted endothelium. Adapted from Totary-Jain et al. 2020 (2).

FIG. 7B are immunofluorescent confocal images illustrating p5RHH-niRFP nanoparticles selectively targeting atherosclerotic plaque regions. Representative confocal en-face images of aorta from human coronary arteries were taken 48 hours after treatment with niRFP mRNA nanoparticles. Images were captured at 60× magnification. Scale bars represents 20 μm. VE-Cadherin to stain ECs, DAPI to stain nuclei. Arrows indicate RFP positive cells at regions of disrupted endothelium. Adapted from Totary-Jain et al. 2020 (2).

FIG. 8A are representative images of a de-identified, non-transplantable human hearts from which coronary arteries were isolated. Hearts are from donors of various ages, medical histories, and cardiovascular disease (CVD) risk factors (e.g., myocardial infarctions, hypertension, and coronary artery disease).

FIGS. 8B-8C are graphic illustrations of total RNA extraction and qPCR for IL-1β mRNA (FIG. 8B) and IL-6 (FIG. 8C) that show increasing values corresponding to increasing age of donor and presence of ASCVD risk factors. RNA was extracted from donors ranging in age from 36-72 years old. The 36 year old donor did not have a history of ASCVD; older donors had various conditions such as atherosclerosis, hypertension, and hyperlipidemia.

FIGS. 8D-8E are graphic illustrations of total RNA extraction and RT-qPCR for IL-1β mRNA (FIG. 8D) and IL-6 (FIG. 8E) demonstrating local inflammation in human coronary arteries. The coronary arteries isolated from donors of various ages and risk factors show increased expression of the pro-inflammatory cytokines. RTqPCR results were normalized to donor H38, a 21-year-old female with no CVD risk factors. IL-1β and IL-6 mRNA expression levels were normalized to GAPDH.

FIG. 8F is a graphic illustration of ex-vivo perfusion experiments and SYBR qPCR for IL-1β normalized to GAPDH to target local inflammation using nanoparticles containing siRNA. Coronary arteries isolated from donor H39 (58 year old male donor, smoker and alcohol use). H39-1 is coronary artery snap frozen and no perfusion treatment. For perfusion H39-14 and H39-15, the coronary arteries, balloon injured 3×, were first perfused with DMEM media containing 5 ng/mL TNF-α/IFN-γ for 4 hr at 10-15 mmHg pressure. After stimulation, H39-14 (control perfusion) artery was perfused with control DMEM media (no nanotherapy treatment), and H39-15 (nanoparticle perfusion, siIL-1β) artery was perfused with DMEM media containing 20 pmol siRNA to siIL-1β (1159) and 40 nmol of p5RHH, for an additional 4 hrs at 10-15 mmHg pressure. After 4 hr perfusion treatment, hCA were collected in normal DMEM media for 24 hr, and then snap frozen.

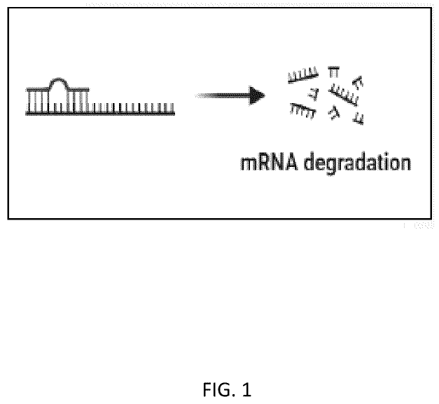

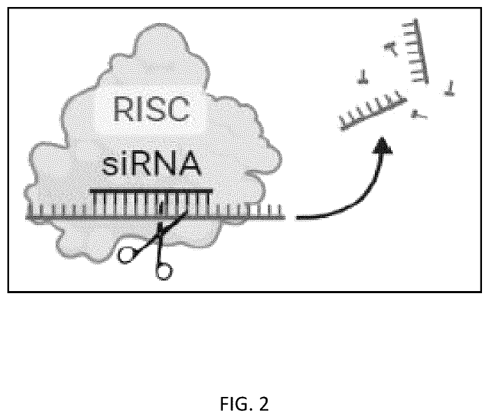

### DETAILED DESCRIPTION

Provided are compositions (e.g., polynucleotides), formulations, and methods for site- and cell-selective therapies. The compositions include RNA molecules that can be introduced in vivo, such as into a subject via a carrier that delivers the RNA molecule to and into cells of the subject. The RNA molecule encodes a protein that prevents, inhibits, or otherwise alleviates symptoms of disease, such as atherosclerosis. The RNA molecule may further include a microRNA (miRNA) target that either inhibits or activates translation of the coding sequence (CDS) when bound by the corresponding miRNA. The RNA molecule may further include a cleavable short interfering (siRNA) sequence that targets an endogenous mRNA or a long non-coding RNA (long ncRNA; lncRNA) in the cell. The carrier may include a polymer, cell penetrating peptide, or lipid-based transfection reagent. Once transferred into the cell en masse, the RNA molecule may be selectively translated in select cells based on the miRNA target sequence, and the siRNA sequence, once cleaved, may target specific RNAs. The compositions, formulations, and methods are of significant use for conditions that require differential modulation of expression between cell types, such as the differential expression of immunomodulating genes between endothelial cells and immune cells near sites of vessel damage. The selective modulation is an improvement over previous methods for controlling expression of endogenous and/or exogenous genes within selective cell-types in vivo.

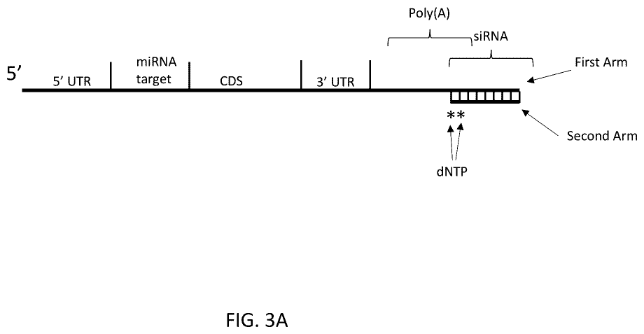

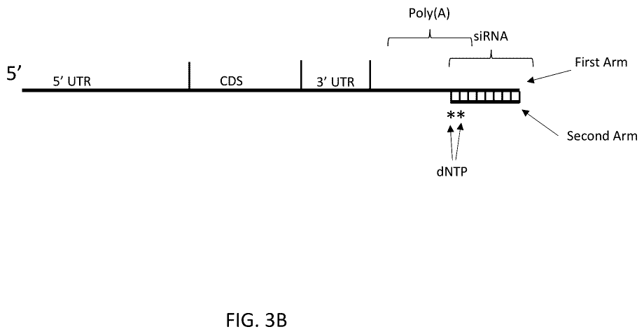

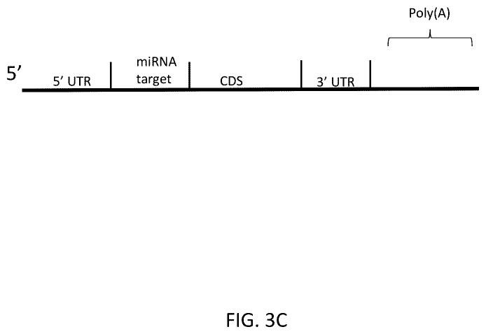

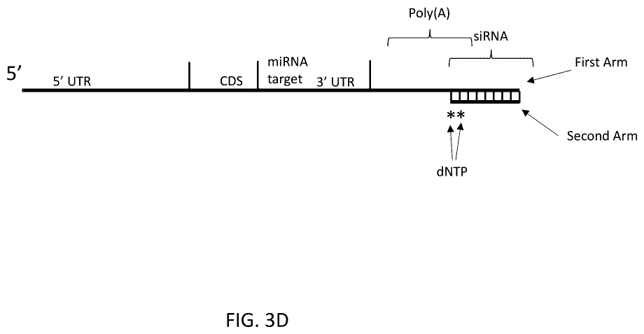

FIG. 3A is an exemplary drawing depicting a polynucleotide operatively used for cell-selective expression of an exogenous gene and modulation of one or more exogenous genes in a subject. The polynucleotide is shown as an RNA molecule; however, the polynucleotide may also be a DNA molecule or construct that is translated into the RNA molecule in FIG. 3A. For reasons of clarity the term “polynucleotide” will be used to indicate either the RNA molecule or the DNA construct used to express the RNA molecule.

The RNA molecule may be synthetically made (e.g., via a synthesizer) or expressed and purified. For example, the RNA molecule may be expressed from a DNA vector, such as a DNA plasmid or linear DNA under the control of a promoter. The promoter may be a phage/viral protein (e.g., T7, T3, SP6), bacterial promoter, or eukaryotic promoter.

As used herein, “promoter” can refer to all nucleotide sequences capable of driving or initiating transcription of a coding or a non-coding DNA sequence. The term “promoter” as used herein can refer to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

Once purified, the RNA molecule may then be encapsulated in a carrier and administered to a subject. In some embodiments, the polynucleotide is included within a vector or virus that expresses the RNA molecule in vivo. For example, the polynucleotide may be cloned into an expression plasmid that is subsequently packaged with a carrier, and administered to a subject. In another example, the polynucleotide may be cloned into viral vector that is subsequently administered.

The subject may be any living organism including a mammal (e.g., human, dog, cat, pig, cattle), reptile, amphibian, fish, or fungi. The subject may also include any living cells, tissues, or organs of any living organism. For example, the subject may include eukaryotic cells grown under tissue culture conditions.

In embodiments, the polynucleotide includes a coding sequence (CDS) for a protein of interest. The protein encoded by the CDS may be of any amino acid sequence and may be a protein whose expression is intended to treat, prevent, and/or alleviate symptoms of disease. Such diseases may include but not limited to heart and vascular disease, cancer, respiratory disease, obesity, neurological disease, diabetes, addiction, infection (e.g., viral, bacterial), and immune-related disease. For example, the CDS may encode a protein designed to target cardiovascular diseases, such as atherosclerotic cardiovascular disease (ASCVD). For instance, the CDS may encode CDKN1B, also known as p27 or Kip1, and vascular endothelial growth factor A (VEGF-A). The CDS may also be a fragment or mutated form of any known protein.

In embodiments, the polynucleotide may include a noncoding RNA, such as a long noncoding RNA (lncRNA). For example, the polynucleotide may include a lncRNA in lieu of the CDS.

In embodiments, the polynucleotide includes a 5′ and/or 3′ untranslated regions (UTR) located upstream and downstream of the CDS, respectively. The UTRs provide stability for the RNA and may also provide a substrate for other polynucleotide elements. The 5′ and 3′ UTRs may be of any sequence, may be sourced/derived from any gene, and may be sourced from a gene different from the gene encoded by the CDS. For example, the 5′ and/or 3′ UTR may be sourced from a β-globin gene, while the CDS may encode the CDKN1B protein.

The polynucleotide may further include a polyadenylate (poly(A)) tail near the 3′ end of the RNA. The poly(A) tail may be of any length. In some aspects, the poly(A) tail may include one of more non-adenine bases. For example, the poly(A) tail may include one or more uridines, cytidines, guanines, non-canonical bases, such as pseudouridine or a pseudouridine analog, or dNTPs such as thymidine, or other DNA bases. Since the poly(A) tail has been shown to increase the stability (i.e., half-life) of the polynucleotide in vivo, the addition of non-adenine bases in the poly(A) tail of a mature polynucleotide (RNA) is designed such that the poly(A) tail still retains some increase of half-life over a similar polynucleotide where the poly(A) tail is removed.

In an aspect, the polynucleotide includes one or more non-canonical bases. The one or more non-canonical bases may be implemented at any point throughout the RNA molecule. The non-canonical bases may include any base or base modification including but not limited to pseudouridine, N1-methylpseudouridine, m6A, m5C, 2′-O-Me, inosine and dNTP. For example, the RNA molecule may include one or more pseudouridines. The inclusion of non-canonical bases into the mRNA, particularly pseudouridine and N1-methylpseudouridine, are expected to increase the stability and translation of the RNA molecule in vivo.

In an aspect, the polynucleotide further includes one or more miRNA target sites that binds one or more miRNAs. The miRNA target site may be operationally linked or otherwise positioned anywhere within the expressed RNA molecule, including but not limited to the 5′ UTR, 3′ UTR, or CDS. The one or more miRNA target sites may bind more than one of the same species of miRNA (e.g., multiple similar sites), or may include different sites (e.g., two miRNA target sites targeted by two different miRNAs. The number of miRNA targets can range from 1 to 20 or more. For example, in some embodiments, the cell-selective RNA molecules can contain 1, 2, 3, 4, or 5 miRNA targets. The miRNA target(s) are nucleotide sequences that can specifically bind one or more miRNAs. The miRNA(s) can have differential spatial and temporal expression. As such, effective expression of the protein encoded by the CDS can be controlled both spatially and temporally depending on the miRNA target(s) included in the cell-selective RNA molecule. For example, binding of a miRNA to the miRNA target sequence may inhibit translation of the CDS of the targeted mRNA.

As used herein, the terms “microRNA” and “miRNA” are used interchangeably, and refer to a small non-coding RNA molecules containing about 21 to about 24 nucleotides. miRNA is found in plants, animals, and some viruses, and functions in transcriptional and post-transcriptional regulation of transcription and translation of RNA. MicroRNA can exist as part of a larger nucleic acid molecule (pri-miRNA, pre-miRNA) such as a stem-loop structure that can be processed by a cell and yield microRNAs of about 21-24 nucleotides.

Suitable miRNA/target pairs include, but are not limited to, miR-126, miR-145, miR-296, miR-21, miR-22, miR-15a, miR-16, miR-19b, miR-92, miR-93, miR-96, miR-130, miR-130b, miR-128, miR-9, miR-125b, miR-131, miR-178, miR-124a, miR-266, miR-103, miR-9*, miR-125a, miR-132, miR-137, miR-139, miR-7, miR-124b, miR-135, miR-153, miR-149, miR-183, miR-190, miR-219, miR-18, miR-19a, miR-24, miR-32, miR-213, miR-20, miR-141, miR-193, miR-200b, miR99a, miR 127, miR-142-a, miR-142-s, miR-151, miR-189b, miR-223, miR-142, miR-122a, miR-152, miR-194, miR-199, miR-215, miR-1b, miR-1d, miR-133, miR-206, miR-208, miR-143, miR-30b, miR-30c, miR-26a, miR-27a, let-7a, and miR-7b. For example, the miRNA target may include a target for miR-126, which is expressed in endothelial cells, but expressed either at low or undetectable levels (e.g., essentially non-expressed) in infiltrating immune cells (e.g., neutrophils, macrophages, monocytes, lymphocytes, and mast cells) and vascular smooth muscle cells (VSMCs). Thus, in endothelial cells, the expressed miRNA will bind to the miR-126 target on the polynucleotide, and regulate (e.g., inhibited or suppress) the expression of the polypeptide encoded by the CDS on the polynucleotide. In contrast, in infiltrating immune cells that do not express miR-126, expression of the CDS will not be suppressed or inhibited, and the CDS will be expressed.

As used herein “miRNA target” or “miRNA target sequence” refers to the nucleic acid sequence, typically RNA, that a miRNA specifically binds to. Thus, the miRNA target comprises or consists of a sequence that is complementary to a selected miRNA. As an example, microRNA 126 (miR-126) can specifically bind a miR-126 target. Binding of a miRNA to a miRNA target can result in transcription and/or translation inhibition of the nucleic acid sequence, such as through degradation of the nucleic acid sequence (typically mRNA or other type of RNA), that the miRNA target is part of. A microRNA does not have to have perfect complementarity to a miRNA target for specific binding or transcription inhibition to occur. The miRNA may further include a seed sequence or seed region. As used herein “seed sequence” or “seed region” refers to the conserved heptametrical sequence of a microRNA that has perfect complementarity to the miRNA target. The seed sequence can be at about positions 2-7 from the miRNA 5′-end.

In some embodiments, the polynucleotide includes a portion of an siRNA duplex (i.e. one arm of a siRNA hybridized to complementary DNA-RNA duplex). In the interest of clarity, an RNA molecule having one or both arms of the siRNA are considered to have an siRNA domain. For example, the RNA molecule may include an siRNA domain that initially includes one arm of the siRNA duplex that is initially transcribed along with the rest of the RNA molecule (i.e., via in vitro transcription (IVT)). After transcription (e.g., in vitro transcription (IVT)), a second arm of the complementary DNA-siRNA hybrid arm may be synthesized and hybridized to the first arm, creating a siRNA domain that now includes a duplex RNA.

Alternatively, the polynucleotide may be designed so that both arms of the duplex are synthesized, creating a hairpin siRNA structure.

The term “operatively coupled” or “operatively linked” as used herein refers to the association of nucleic acid sequences on a single nucleic acid fragment so that the function of one is regulated by the other. For example, a promoter is operatively linked with a coding sequence when it is capable of regulating the expression of that coding sequence (i.e., the coding sequence is under the transcriptional control of the promoter). Coding sequences can be operatively linked to regulatory sequences in a sense or antisense orientation. In one example, the complementary RNA regions can be operatively linked, either directly or indirectly, 5′ to the target mRNA, or 3′ to the target mRNA, or within the target mRNA, or a first complementary region is 5′ and its complement is 3′ to the target mRNA. The term “operatively linked” as used herein can also refer to the direct or indirect linkage of any two nucleic acid sequences on a singly nucleic acid fragment such that they are indirectly or directly physically connected on the same nucleic acid fragment. The term “operatively linked” as used herein can also refer to the insertion of a nucleic acid within the 5′ and 3′ end of another nucleic or the direct coupling of a nucleic acid to the 5′ or 3′ end of another nucleic acid.

In an aspect, the RNA molecule may include one or more DNA nucleotides at or near the siRNA duplex that promotes cleavage of the siRNA duplex in vivo. For example, nucleases, such as RNase H, cleave RNA-DNA hybrids. These hybrids may be produced via the addition of dNTPs to the RNA molecule, whether through extension of the RNA molecule (e.g., via reverse transcriptase acting on the hairpin at the 3′ end of the RNA molecule), or through hybridization of a DNA-RNA oligonucleotide to the RNA molecule. Once generated and introduced into the cell, endogenous RNase H recognizes the hybrid nature of the polynucleotide and cleaves the polynucleotide at or near the DNA bases, creating a double-strand break that releases the siRNA from the polynucleotide. Once the siRNA is cleaved from the polynucleotide, the siRNA may then be processed further to form an RNA-Induced Silencing Complex (RISC). A single-stranded region within the RISC then binds an endogenous mRNA target, creating a dsRNA region that is then cleaved by endogenous nucleases, degrading the mRNA target.

In an aspect, the siRNA may be designed to target any endogenous gene. For example, the siRNA may be designed to target genes involved in diseases including but not limited to heart and vascular disease, cancer, respiratory disease, obesity, neurological disease, diabetes, addiction, infection (e.g., viral, bacterial), and immune-related disease. For example, the siRNA may be designed to target cardiovascular diseases, such as atherosclerotic cardiovascular disease (ASCVD). For instance, the siRNA may be designed to target inflammation modulators in ASCVD including but not limited to Interleukin 1 (IL-1) isoforms such as IL-1α and IL-1β, and interleukin 6 (IL-6).

In some embodiments, the polynucleotide may include the capability to produce multiple siRNA duplexes. For example, the in vitro transcribed RNA molecule may include several first arms of siRNA duplexes that are then hybridized to several second arms, each containing DNA bases recognizable by RNase H. Once introduced into the cell, endogenous RNAse H may cleave and release several siRNA duplexes from one polynucleotide molecule.

Once cleaved, the siRNA may have the form of any operative siRNA molecule. For example, the siRNA may include a hairpin structure, or be constructed of two complementary oligonucleotides. The siRNA may be of any operative length for the cleavage of an mRNA target. For example, the cleaved siRNA molecule may be 17, 18, 19, 20, 21, 22, 23, 24, or 25 nucleotides in length.

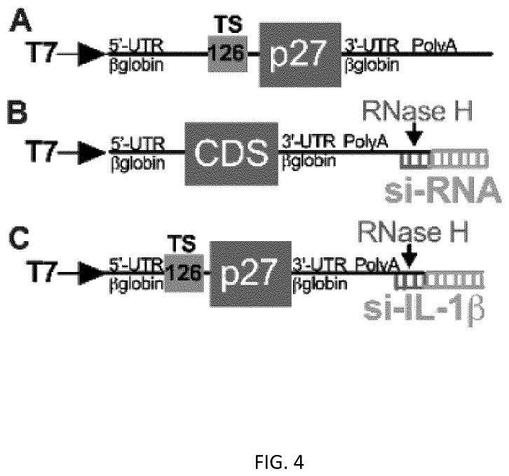

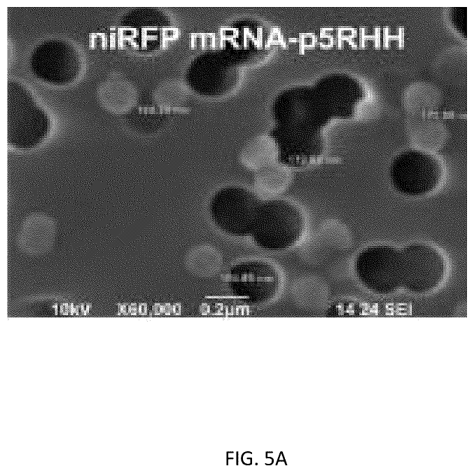

FIGS. 3B to 3D and FIGS. 4A-C illustrate various nonlimiting configurations of the polynucleotide. For example, the polynucleotide may include an RNA molecule without a miRNA target sequence (FIG. 3B). In another example, the polynucleotide may include an RNA molecule without an siRNA sequence (FIG. 3C and FIG. 4A). In another example, the polynucleotide may include an RNA molecule with the miRNA target in the 3′ UTR region (FIG. 3D).

In an aspect, a formulation is disclosed that includes the polynucleotide and a carrier that delivers the polynucleotide into a cell. The carrier may be any type of polynucleotide transferring medium including but not limited to lipids (e.g., for transfections), precipitating salts (e.g., calcium phosphate), polymers, and cell-penetrating peptides. For example, the carrier may include the cell-penetrating peptide p5RHH having the amino acid sequence VLTTGLPALISWIRRRHRRHC (SEQ ID NO.1). The carrier may include a mixture of carrier media. For example, the carrier may contain both lipids and cell-penetrating peptides, or polymers and cell-penetrating peptides.

As used herein, the term “transfection” can refer to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be associated with various proteins or regulatory elements (e.g., a promoter and/or signal element, miRNA target sequences as described herein), or the nucleic acid may be incorporated into a vector or a chromosome.

In another aspect, a method is disclosed for treating or preventing a disease, or alleviating/preventing symptoms of a disease, such as ASCVD, or any other disease or condition as described herein, using the disclosed polynucleotide. For example, the method may include preventing, inhibiting, or alleviating vessel stenosis. In another example, the method may include preventing, inhibiting, or alleviating inflammation and/or atherosclerotic plaque formation.

In an embodiment, the method includes administering to a subject the formulation described herein. As used herein, “administering” can refer to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, by catheters, balloon angioplasty, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

The term “treating”, as used herein, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

In another embodiment, the method includes obtaining the formulation (e.g., from a vendor), or through production of one or more constituents of the formulation, or through mixture of one or more constituents of the formulation. For example, a commercial formulation of may include a premixed polynucleotide and carrier that can be administered to a subject. In another example, the formulation is obtained as separate constituents. For instance, polynucleotide and carrier constituents may be obtained in separate packaging (e.g., vials) that that are mixed, which incorporates the polynucleotide into the carrier before administration. The formulation may further include one or more diluents that are added to the formulation or constituents of the formulation before or after incorporation of the polynucleotide into the carrier.

In an embodiment, the method includes incorporating the polynucleotide into a carrier. For example, incorporating the polynucleotide into the carrier may include simple mixing (e.g., repeated inversion of a mixture in a vial) of the polynucleotide with the carrier. In another example, incorporating the polynucleotide into the carrier may include other methods including but not limited to vortexing and sonicating. The incorporation of the polynucleotide into the carrier may be obtained with or without the addition of diluents.

In embodiments, the method includes incorporating the polynucleotide into a carrier to obtain the formulation, and administering the formulation to a subject. For example, the formulation may be administered to a subject for treating or preventing a disease, or alleviating/preventing symptoms of a disease, such as ASCVD, or any other disease or condition as described herein. Any form of administration as described herein may be used to deliver the formulation to the subject.

As used herein, “polypeptides” or “proteins” are amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “gene” can refer to a hereditary unit corresponding to a sequence of DNA that occupies a specific location on a chromosome and that contains the genetic instruction for a characteristic(s) or trait(s) in an organism. “Gene” also refers to the specific sequence of DNA that is transcribed into an RNA transcript that can be translated into a polypeptide or be a catalytic RNA molecule including but not limited to tRNA, siRNA, piRNA, miRNA, long-non-coding RNA and shRNA.

As used herein, “deoxyribonucleic acid (DNA)” and “ribonucleic acid (RNA)” generally refer to any polyribonucleotide or polydeoxribonucleotide, which can be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers or coding mRNA (messenger RNA).

As used herein, “nucleic acid sequence” and “oligonucleotide” also encompasses a nucleic acid and polynucleotide as defined elsewhere herein.

As used herein, “DNA molecule” includes nucleic acids/polynucleotides that are made of DNA.

As used herein, “nucleic acid” and “polynucleotide” generally refer to a string of at least two base-sugar-phosphate combinations and refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. The strands in such regions can be from the same molecule or from different molecules. The regions can include all or one or more of the molecules, but more typically involve only a region of some of the molecules. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotide” as that term is intended herein.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein with reference to the relationship between DNA, cDNA, CRNA, RNA, and protein/peptides, “corresponding to” can refer to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, the term “exogenous DNA” or “exogenous RNA” or exogenous nucleic acid sequence” or “exogenous polynucleotide” can refer to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

Nano-therapy has been developed consisting of self-assembled cell-penetrating peptides (e.g., p5RHH) and a microRNA (miRNA) switch encoding the cell cycle inhibitor, p27, with a binding site for the endothelial cell (EC) specific miR-126 at its 5′UTR. Administration of this nano-therapy to a wire injury mouse model showed it specifically targeted endothelial denuded regions, reduced neointima formation, and allowed re-endothelialization. Therefore, the overall goal of this disclosure is to advance this site- and cell-selective therapy by targeting both cell proliferation and inflammation that contribute to plaque formation, while preserving the vasculo-protective endothelium.

Research Significance. ASCVDs are the leading cause of death in the United States and impart a significant healthcare burden on the nation. Current medications treat risk factors such as hypercholesterolemia and hypertension, but therapies that specifically target the cells that build atherosclerotic plaques while sparing the endothelium are not available. A competent endothelium is vital to maintaining vessel health, as this monolayer of cells lining the arterial intima secretes endogenous vasodilators like nitric oxide and performs many anti-inflammatory and anti-thrombotic functions. Furthermore, the role of inflammation in atherosclerosis has been appreciated for years, but only recently clinically evaluated as a therapeutic target.

Interleukin 1 (IL-1) is a mediator of systemic inflammation, released from activated NLRP3 inflammasomes; it exists in two isoforms, IL-1α and IL-1β. NLRP3 is highly expressed in the plaques of patients with atherosclerosis, and greater expression has been correlated with atherosclerosis severity. Additionally, oxidized LDL and cholesterol crystals, key features of plaques, trigger inflammasome activation, leading to increased release of IL-1β, creating an amplification loop that increases interleukin 6 (IL-6) production from ECs, VSMCs, and macrophages. IL-6 has been implicated in atherothrombosis, increased VSMC proliferation, and pro-inflammatory functions of ECs, making IL-1β a promising therapeutic target to stop this amplification process and atherosclerosis progression.

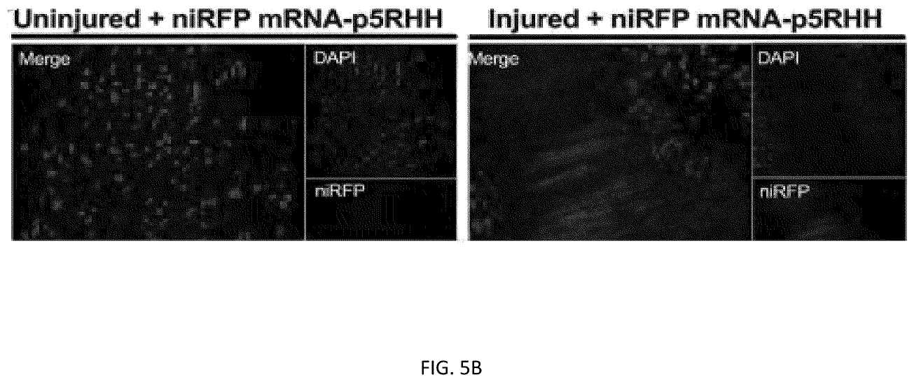

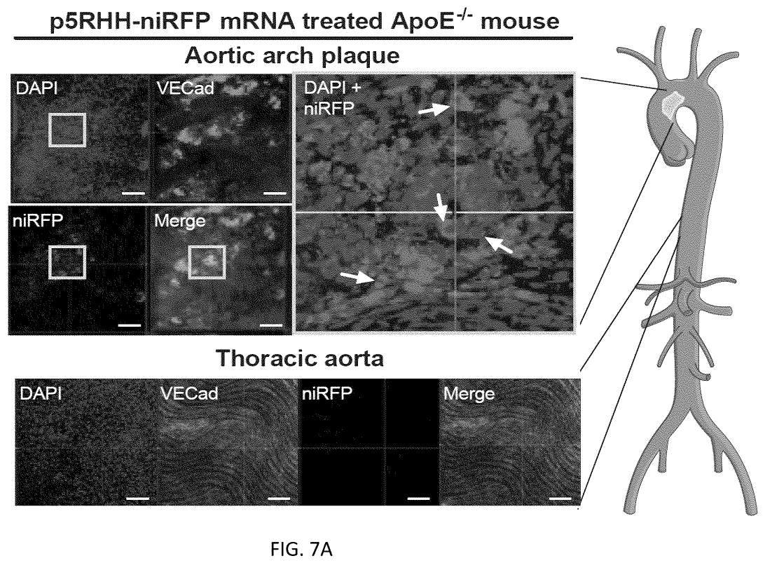

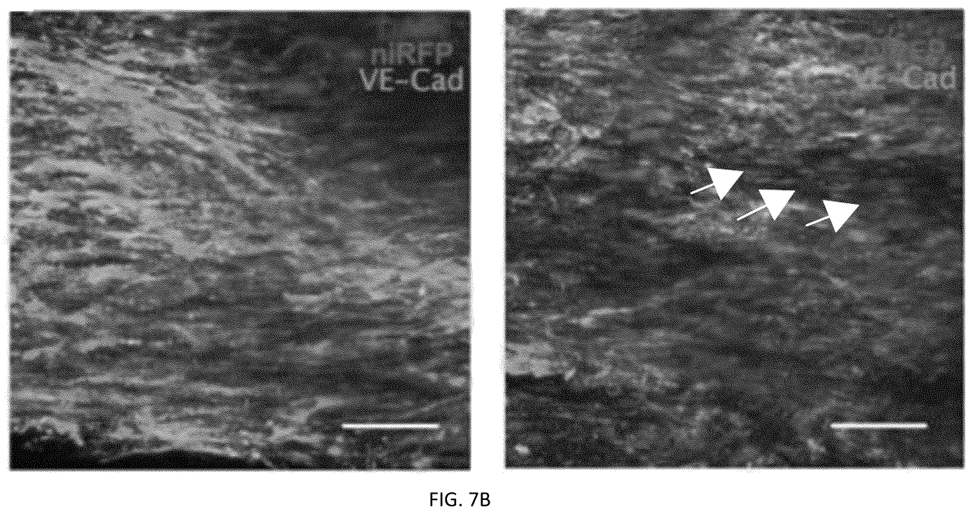

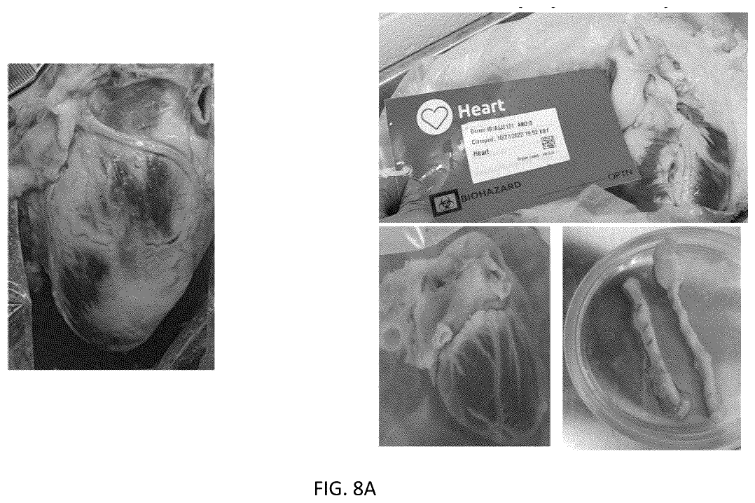

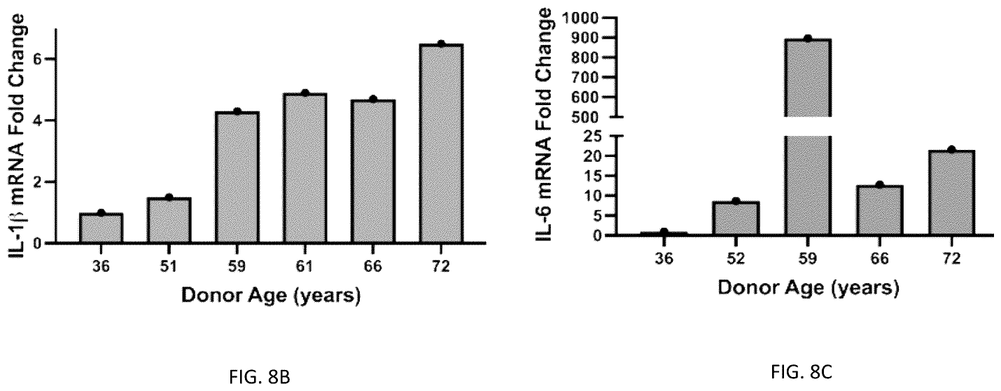

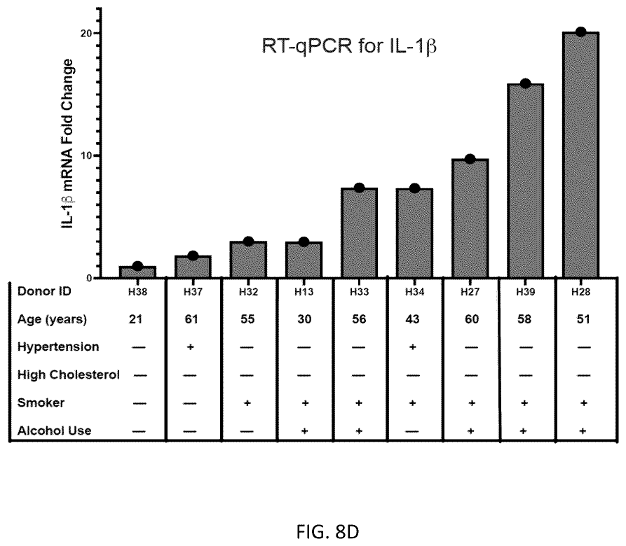

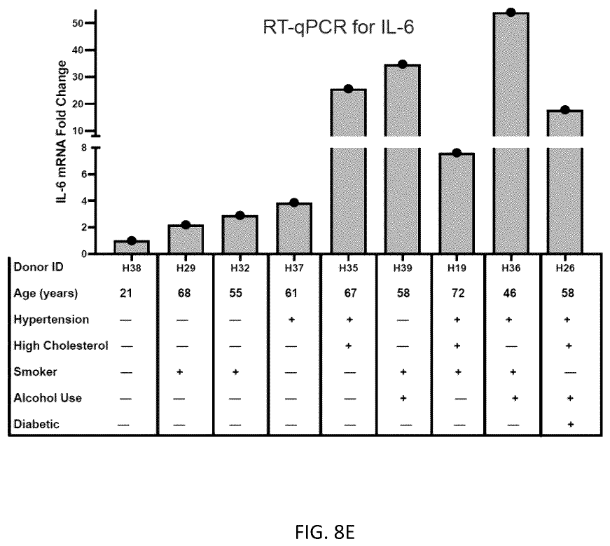

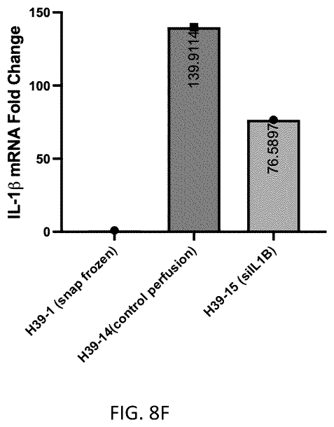

The groundbreaking clinical trial, CANTOS, leveraged a monoclonal antibody against IL-1β to treat patients with a history of myocardial infarctions, and reported decreased recurrent cardiac events compared to placebo. However, patients also had an increased risk of infections from the systemic immune inhibition, demonstrating the need to specifically target inflammation only in the plaques. Thus, in this application, an inventive site- and cell-selective mRNA-based nano-therapy is to be developed in which several cutting edge techniques will be combined into one single mRNA therapeutic: 1) miRNA switch consisting of synthetic mRNA encoding the cell cycle inhibitor, p27, with one complementary target site (TS) for the EC-specific miR-126 in its 5′UTR, allowing for selective expression of p27 in VSMCs and infiltrating immune cells, while protecting the endothelium, as endogenous expression of miR-126 in ECs will inhibit its expression (FIG. 4A); 2) combined hybrid structure of siRNA tailed mRNA (ChriST mRNA) that consists of an siRNA sequence to IL-1β encoded in its poly(A) tail, annealed with a complementary strand of RNA-DNA hybrid, which is then cleaved by RNase H upon intracellular delivery to release the siRNA (20) (FIG. 4B). Combination of the p27-miRNA switch with an IL-1β siRNA into a single mRNA therapeutic (FIG. 4C) will be a more potent anti-atherosclerotic therapy than each individually. As a delivery method, the cell-penetrating peptide, p5RHH, will self-assemble with the synthetic mRNA into compact nanoparticles (FIG. 5A) and allow for site-specific delivery only to disrupted regions of endothelium (FIG. 5B and FIGS. 7A-7B).

The American Heart Association's Impact Goal for 2030 is to increase the life expectancy of individuals in the US and worldwide. The main objective of this research aligns with that mission. A novel anti-atherosclerotic therapeutic will be developed that will revolutionize the way CVDs are treated and expectantly decrease mortality rates associated with them, allowing people to live longer, healthier lives.

### Example 1 Preliminary Studies

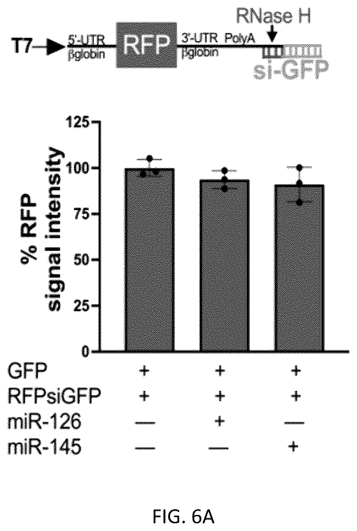

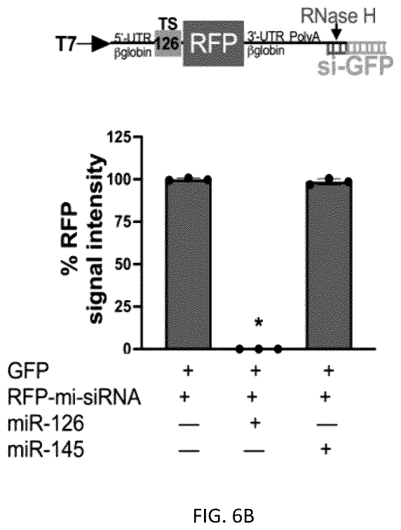

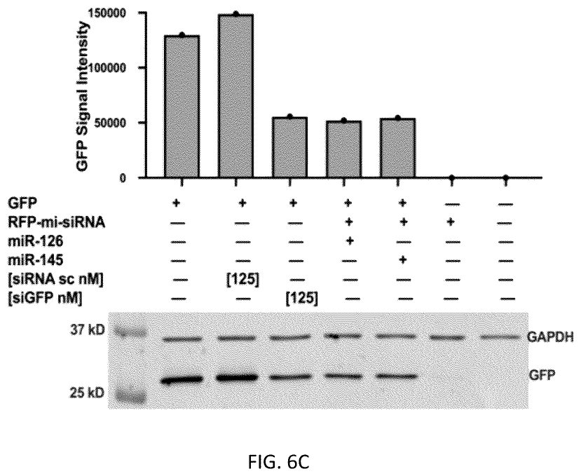

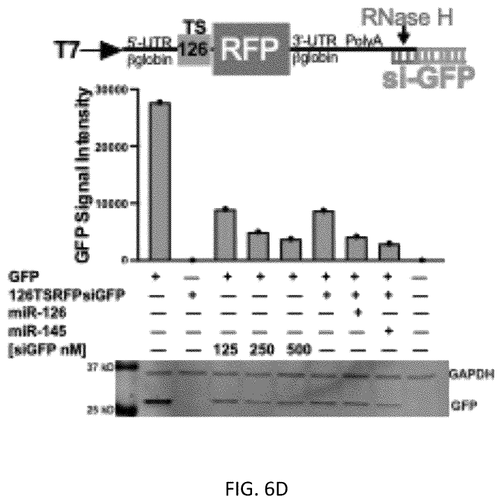

To provide proof of the feasibility to combine miRNA switch and ChriST technologies into a single mRNA construct, a mRNA encoding red fluorescent protein (RFP) with no TS or with one miR-126 TS in its 5′ UTR, and an siRNA to target green fluorescent protein (GFP) in their poly(A) tails were in vitro transcribed (IVT), and designated RFP-siGFP (FIG. 6A) and 126TS-RFP-siGFP (FIGS. 6B and 6D), respectively. To test the efficacy of the miRNA switch, and since 293T cells do not express miR-126, cells were transfected with miRNA mimics of either miR-126 or miR-145 (control). After 24 hours, cells were co-transfected with GFP mRNA and either RFP-siGFP or 126TS-RFP-siGFP mRNA. Flow cytometry showed that in cells transfected with RFP-siGFP, RFP expression was not affected by miR-126 or miR-145 (FIG. 6A), whereas cells transfected with 126TS-RFP-siGFP abolished RFP expression in the presence of miR-126 but not miR-145 mimics (FIG. 6B). Moreover, cells transfected with 126TS-RFP-siGFP mRNA also exhibited a decrease in GFP expression, which was comparable to 125 nM of standard siGFP treatment (FIG. 6D). These preliminary studies provide proof of the feasibility of the nano-therapy design with the potential to reduce aberrant proliferation of both VSMCs and infiltrating immune cells, as well as decreasing inflammation associated with atherosclerotic plaques, all while sparing the endothelium to preserve its protective properties.

### Example 2 Design and Test the Efficacy of a Nano-Therapy Containing a Combined p27-miRNA Switch and Interleukin-1β (IL-1β) siRNA In A Single mRNA Construct.

Vascular smooth muscle cells (VSMCs) and macrophages contribute to atherosclerosis progression via their aberrant cell proliferation and inflammatory cytokine release. In a prophetic example, a novel mRNA-based therapeutic is designed combining the p27-miRNA switch with an siRNA to IL-1β in its poly(A) tail. Transfection of this synthetic mRNA to ECs, VSMCs, and monocyte derived macrophages are expected to decrease inflammatory cytokine release from all cell types, and proliferation inhibition in VSMCs and macrophages, but not ECs.

Approach: Design and test the efficacy of a nanotherapy containing a combined p27-miRNA switch and interleukin-1B (IL-1B) siRNA in a single mRNA construct.

Background and Rationale: p27 is a cyclin-dependent kinase inhibitor that can arrest cells in G1 phase of the cell cycle; upregulation of p27 has been shown to significantly reduce the development of neointima formation after vascular injury. Additionally, macrophages, ECs, and VSMCs of atherosclerotic plaques release pro-inflammatory cytokines that contribute to atherosclerosis progression. Combining miRNA switch technology to confer cell-selectivity in expressing p27 in only VSMCs and macrophages, while sparing ECs, with ChriST technology to inhibit IL-1β and inflammation, is expected to yield a multi-function therapeutic in a single mRNA construct.

Experimental Design: a mRNA will be designed encoding p27 with a single binding site for the EC specific miR-126 in its 5′UTR and an siRNA to IL-1β in its poly(A) tail (126TS-p27-siIL-1β, FIG. 4C). The mRNA will be IVT using 100% ψ substitution to lessen immunogenicity and 5′ capped. Experimental groups of human ECs, VSMCs, and monocyte derived macrophage cell cultures will be transfected with 126TS-p27-siIL-1β using Lipofectamine 2000. Control groups will be transfected with a mRNA construct encoding RFP and containing a scramble miRNA target site in its 5′ UTR and encoding a scramble siRNA sequence in its poly(A) tail, annealed with a complementary siRNA sequence (scramble-RFP). Expression of p27 will be evaluated via Western blot, while IL-1β and IL-6 cytokine levels will be measured via ELISA of the cell culture supernatant after transfection. Cell proliferation will be measured via MTT assay.

It is expected that VSMCs and macrophages transfected with 126TS-p27-siIL-1β will exhibit increased p27 expression and decreased proliferation compared to scramble-RFP (control) transfected cells, whereas ECs will not exhibit an increase in p27 expression because they endogenously express miR-126 and therefore will continue to proliferate. However, IL-1β and IL-6 levels are expected to decrease in all experimental groups transfected with 126TS-p27-siIL-1β, as miR-126 will not impede the RNase H dependent release of the siRNA. Separate delivery of a p27-miRNA switch mRNA and siRNA to inhibit IL-1β will be feasible to test any issues. In order to differentiate between endogenous p27 expression and expression from 126TS-p27-siIL-1β, mRNA will be designed to encode a FLAG tag. Additionally, if IL-1β and IL-6 secretions are not detected in the cell cultures before transfection, NLRP3 inflammasome activation will be induced by first treating cells with lipopolysaccharide (LPS) before experimentation.

### Example 3 Evaluate Plaque Regression, Inflammation, Vessel Healing, and Translational Capacity of the Combined p27-miRNA Switch and IL-1β siRNA mRNA.

In order to test the effectiveness of this nano-therapy in causing plaque regression, the combined p27-miRNA switch-IL-1β siRNA will be delivered to the ApoE-/- mouse model of atherosclerosis, using p5RHH to specifically target the combination siRNA to disrupted regions of the endothelium. Mice treated with this therapeutic are expected to have greater plaque regression, decreased inflammation, and increased healing to the endothelium, compared to controls. Additionally, in order to bring this approach closer to clinical relevance, human coronary arteries isolated from donated, non-transplantable human hearts will be perfused with this nano-therapy, and the ability of the nano-therapy to be targeted to regions of atherosclerotic plaques, in donors with histories of ASCVDs evaluated.

Approach: Evaluate plaque regression, inflammation, vessel healing, and translational capacity of the combined p27-miRNA switch and IL-1β siRNA mRNA.

Background and Rationale: It was previously shown above that delivery of a p27-miRNA switch to a wire injury mouse model resulted in neointima reduction. Therefore, delivery of 126TS-p27-siIL-1β mRNA using p5RHH in vivo to ApoE-/- mice is expected to allow for delivery to atherosclerotic plaques only and not to regions of intact endothelium (FIG. 7A), and lead to plaque regression, decreased inflammation, and accelerated vessel healing. Furthermore, to test delivery to human tissue and assess translational potential, human coronary arteries containing atherosclerotic plaques will be perfused with the nano-therapy in an ex vivo model (FIG. 7B).

Experimental Design: In vivo ApoE-/- mouse model to evaluate plaque regression, inflammation, and endothelium integrity: 6-week-old ApoE-/- mice will be fed a high fat western diet for 15 weeks prior to the beginning of experimentation to allow sufficient time for plaque formation and maintained on this diet throughout the study. Mice will receive IV administrations every 3 days for 4 weeks (10 total injections) of nanoparticles composed of p5RHH and either 100% ψ modified (1) 126TS-p27-siI-L1β mRNA or (2) niRFP mRNA as control. To assess atherosclerosis progression, mouse aortas will be dissected, fixed, and stained with Oil Red O solution to stain lipoproteins and quantify the extent of the atherosclerotic plaques. Additionally, hematoxylin and eosin (HE) staining will be done on cross sections of isolated arteries to determine intima-to-media (I/M) area ratios. Image analysis will be done using ImageJ Software.

To visualize cells of the atherosclerotic plaques, immunostaining will be done with α-smooth muscle actin to visualize VSMCs, anti-CD45 and anti-CD68 to visualize immune cells and macrophages, respectively, and anti-CD31 to visualize endothelial cells, which will also be used to determine endothelium integrity. To assess distribution of 126TS-p27-siIL-1β, aortas, kidneys, lungs, liver, and spleens of the mice will be collected at the end of the 4-week treatment, and total RNA will be extracted and qRT-PCR as well as Western blot analysis will be done to determine the presence of 126TS-p27-siIL-1β or niRFP mRNA or protein in any of these organs. Finally, to evaluate inflammation, serum samples from live mice will be collected before treatment and after 2 and 4 weeks of treatment to measure levels of circulating IL-1β and IL-6.

Experimental Design: Ex vivo model to deliver nano-therapy to human tissue: To bring this research closer to clinical relevance, an ex vivo model will be used in which coronary arteries will be collected from de-identified, non-transplantable, viable human hearts (donated through the LifeLink Foundation), many from older donors with histories of ASCVDs. Experiments will be conducted to determine the optimal perfusion protocol to treat the arteries. Either sham or balloon injury to the vessels will be performed, flush them with cell culture media (DMEM, with 10% FBS, 2% penicillin/streptomycin), cannulate and tie them to an oxygenated perfusion system, and treat with nanoparticles composed of p5RHH and niRFP to determine the best pressure, dosage, and number of administrations of RNA needed for effective delivery. qRT-PCR and Western blots analysis will be performed for niRFP to determine uptake by the arteries. After an optimal perfusion protocol is determined, arteries will be perfused with nanoparticles composed of p5RHH and either (1) niRFP (control) or (2) 126TS-p27-siIL-1βmRNA. At the end of the perfusion treatments, immunostaining of arterial cross-sections will be done as described in the in vivo study in order to visualize the cells that contribute to the lesions. qRT-PCR and Western blot analysis of the arteries will be done to determine p27 mRNA and protein expression, as well as qRT-PCR for IL-1β and IL-6.

Successful delivery of the RNA therapeutic in nanoparticle delivery has been shown for both in vivo and ex vivo models (FIG. 7A-7B). For the in vivo model, a reduction in the plaque burden and decreased I/M ratios and inflammatory cytokine levels is expected, as well as greater endothelium integrity in ApoE-/- mice treated with nanoparticles containing 126TS-p27-siIL-1β, compared to controls. Additionally, significant distribution of the mRNA or protein to internal organs of the mice is not expected, as p5RHH should specifically target the nano-therapy to disrupted endothelium only. For the ex vivo model, nanoparticle delivery to human coronary arteries with disrupted endothelium is expected, in donors with histories of atherosclerosis. Also, decreased IL-1β and IL-6 mRNA levels in arteries treated with 126TS-p27-siIL-1β is expected. Additional experiments can be conducted in another set of ApoE-/- mice and delivery of mRNA encoding vascular endothelial growth factor A (VEGF-A) in a nanoparticle, using p5RHH, in addition to the administration of the 126TS-p27-siIL-1β mRNA. Delivery of VEGF-A is expected to stimulate healing of the damaged endothelium. Although previous therapies have been made that are anti-VEGF to treat atherosclerosis, as a concern that VEGF can stimulate neovascularization and lead to plaque rupture, because the p5RHH nanoparticle will deliver VEGF-A to regions of disrupted endothelium only, aberrant neovascularization are not expected to occur. Also, the self-replicating Venezuelan equine encephalitis (VEE) may be used to design a 126TS-p27-siIL-1β mRNA that will maintain its mRNA expression within cells long enough in cells to induce sufficient plaque regression. In order to optimize the experiments to the model being used, mouse p27 will be used for the in vivo experiments and human p27 for ex vivo. Data generated from the ex vivo experiments will be analyzed taking into account each donor's medical history.

Sample Sizes and Statistical Analyses: All in vitro cell culture experiments will be performed in triplicate and repeated at least 3 times. For both the in-vivo and ex-vivo experiments, the sample size of each control and experimental group will be n=6, to achieve a power of 0.868. Comparisons of two groups will be analyzed using an unpaired, one-tailed Student's t-test. For comparisons of three or more groups, a One-way ANOVA analysis will be conducted, followed by a Tukey post hoc test. If data generated does not pass normality tests, then the non-parametric Mann Whitney U test (comparisons of two groups) or a Kruskal-Wallis test (comparisons of three or more groups) will be used. A p<0.05 will be considered significant, and all statistical analyses will be conducted using Prism 8 Software.

### REFERENCES

1. Lockhart J H, VanWye J, Banerjee R, Wickline S A, Pan H, Totary-Jain H. Self-assembled miRNA-switch nanoparticles target denuded regions and prevent restenosis. Mol Ther. 2021 May 5;29(5):1744-1757. doi: 10.1016/j.ymthe.2021.01.032. Epub 2021 Feb. 3. PMID: 33545360; PMCID: PMC8116603.
2. Hana Totary-Jain, John Lockhart, Samuel Wickline and Hua Pan. Abstract 130: Design and Delivery of MicroRNA Switches to Treat Cardiovascular Diseases. Arteriosclerosis, Thrombosis, and Vascular Biology, 2020;40:A130 (Abstract, originally published 29 Jun. 2020).
3. Ridker, P. M., B. M. Everett, T. Thuren, J. G. MacFadyen, W. H. Chang, C. Ballantyne, F. Fonseca, J. Nicolau, W. Koenig, et al., Antiinflammatory Therapy with Canakinumab for Atherosclerotic Disease. New England Journal of Medicine, 2017. 377(12).
4. Ridker, P. M., et al., IL-6 inhibition with ziltivekimab in patients at high atherosclerotic risk (RESCUE): a double-blind, randomised, placebo-controlled, phase 2 trial. The Lancet, 2021. 397 (10289): p. 2060-2069.
5. Lee, K., Kim, T S, Seo, Y., Kim, S Y, and Lee, H., Combined hybrid structure of siRNA tailed IVT mRNA (ChriST mRNA) for enhancing DC maturation and subsequent anticancer T cell immunity. J Controlled Release, 2020; 327:225-234.

1. A polynucleotide comprising:
an RNA molecule comprising:
a coding sequence (CDS), wherein the CDS encodes a protein;
a 3′ untranslated region (UTR) and/or a poly(A) tail;
a microRNA (miRNA) target sequence operatively linked to the coding sequence;
a first arm sequence of a small interfering RNA (siRNA) sequence linked to the 3′ (UTR) or the poly(A) tail by a cleavable linker; and
a second arm of the siRNA sequence.

2. The polynucleotide of claim 1, wherein the protein comprises CDKN1B or VEGF-A.

3. The polynucleotide of claim 1, wherein the miRNA target sequence is a target for miR-126.

4. The polynucleotide of claim 1, wherein the siRNA sequence targets an mRNA for Interleukin-1 beta (IL-1β).

5. The polynucleotide of claim 1, wherein the second arm of the siRNA is at least partially complimentary to the first arm.

6. The polynucleotide of claim 5, wherein the siRNA sequence is a substrate for ribonuclease H (RNase H), wherein cleavage of the substrate by RNase H releases an siRNA duplex from the RNA molecule.

7. A formulation comprising:
the polynucleotide of claim 1; and
a carrier operatively configured to deliver the polynucleotide into cell.

8. The formulation of claim 7, wherein the carrier is selected from the group consisting of a lipid, a precipitating salt, a polymer, and a cell-penetrating peptide.

9. The formulation of claim 8, wherein the carrier comprises a cell-penetrating peptide.

10. The formulation of claim 9, wherein the cell penetrating peptide comprises SEQ ID NO: 1.

11. A method comprising:
obtaining the formulation of claim 7; and
administering the formulation to a subject in need thereof.

12. The method of claim 11, wherein the formulation prevents, inhibits or alleviates vessel stenosis.

13. The method of claim 11, wherein the formulation prevents, inhibits, or alleviates inflammation or plaque formation.

14. A method comprising:
obtaining the polynucleotide of claim 1; and
incorporating the polynucleotide into a carrier.

15. The method of claim 14, wherein the carrier is selected from the group consisting of a lipid, a precipitating salt, a polymer, and a cell-penetrating peptide.

16. The method of claim 14, wherein binding of a miRNA to the miRNA target sequence inhibits translation of the CDS.

17. The method of claim 14, wherein polynucleotide incorporated into the carrier is administered to a subject at risk of or having vessel stenosis.

18. The method of claim 17, wherein polynucleotide prevent, inhibits, or alleviates the vessel stenosis


SEQUENCE LISTING (1 sequence)

SEQ ID NO: 1 - protein, 21 aa, synthetic construct
VLTTGLPALISWIRRRHRRHC